(19) United States
(12) Patent Application Publication
(10) Pub. No.: US 2021/0315809 A1
(43) Pub. Date: Oct. 14, 2021

(54) ORAL CARE COMPOSITIONS AND METHODS

(71) Applicant: Per Os Biosciences, LLC, Hunt Valley, MD (US)

(72) Inventors: Robert Estey, Hunt Valley, MD (US); Lisa Bachmann, Forest Hill, MD (US); William O. Brisben, Hobe Sound, FL (US)

(21) Appl. No.: 17/227,978

(22) Filed: Apr. 12, 2021

Related U.S. Application Data
(60) Provisional application No. 63/009,138, filed on Apr. 13, 2020.

(51) Int. Cl. A61K 9/68; A61K 47/46; A61K 47/26; A61K 47/14; A61K 47/38; A61K 45/06; A61K 31/722; A61K 36/73; A61P 29/00

(52) U.S. Cl. CPC A61K 9/0058; A61K 47/46; A61K 47/26; A61K 47/14; A61P 29/00; A61K 45/06; A61K 31/722; A61K 36/73; A61K 47/38

(57) ABSTRACT

Oral formulations include a polysaccharide, such as, chitosan and an anthocyanin-rich extract from dark berries. These oral formulations are manufactured in various permutations such as a chewing gum or lozenge which incorporate chitosan and berry extract powder for protecting the oral cavity from the bacteria that cause gingivitis and periodontitis.

### CROSS REFERENCE TO RELATED APPLICATIONS

This application claims the benefit of U.S. Provisional Application 63/009,138 filed on Apr. 13, 2020. The entire contents of this application is incorporated herein by reference in its entirety.

### FIELD

The present disclosure relates to the composition and manufacture of an oral tablet containing the polysaccharide chitosan and an anthocyanin-rich extract from dark berries and, more specifically, a chewing gum incorporating chitosan and berry extract powder that protects the oral cavity from the bacteria that cause gingivitis and periodontitis. A further embodiment incorporates one or more cannabinoids as anti-microbial agents and to help reduce inflammation of the gums. A further embodiment incorporates one or more remineralization agents like hydroxyapatite to strengthen tooth enamel.

### BACKGROUND

Gingivitis and periodontal diseases are very common in about 50% of people over 30 years of age, estimated at over 100 million people. These diseases can be prevented and managed through good daily oral hygiene practices and regular visits to the dentist. However, poor compliance continues to result in a relatively poor state of dental health for many adults.

The current oral hygiene therapies to prevent gingivitis suffer from poor compliance (flossing, toothbrushing, oral rinsing) or lack of accessibility and high cost (visits to the dentist and prescription drugs like chlorhexidine).

### SUMMARY

There is an unmet need for a safe, cost-effective anti-gingivitis product that helps people be compliant by using a product that can be easily incorporated into their daily routines. Accordingly, embodiments are directed to oral compositions which deliver therapeutically effective amounts of chitosan and berry extracts to a subject in need thereof. In particular, the oral compositions comprise chewing gum and lozenges.

Accordingly, in certain embodiments, a composition comprising: an active ingredient, a sugar alcohol, a blend of sugar alcohols, a sweetener, flavorings, a gum base, or combinations thereof. In certain embodiments, the composition of claim 1, wherein the active ingredient is an anti-microbial ingredient or combination of anti-microbial ingredients. In certain embodiments, the anti-microbial ingredients comprise chitosan, berry extract(s) or a combination thereof. In certain embodiments, the anti-microbial ingredients comprise from about 1.0% to about 10.0% by weight, based on the total weight of the composition. In certain embodiments, the composition further comprises a cannabinoid, combinations of cannabinoids or derivatives thereof. In certain embodiments, the cannabinoid(s) or derivatives thereof comprise from about 1.0% to about 10.0% by weight, based on the total weight of the composition. In certain embodiments, the composition further comprises a remineralization compound, combinations of remineralization compounds or derivatives thereof. In certain embodiments, the remineralization compound or derivatives thereof comprise from about 1.0% to about 10.0% by weight, based on the total weight of the composition. In certain embodiments, the composition, based on the total weight of the composition, comprises: about 40% to about 80% by weight of a sugar alcohol or a blend of sugar alcohols, or a sweetener or a combination thereof; about 20.0% to about 30.0% by weight of a gum base; about 2% to about 15% by weight of a flavoring in liquid or powder form; about 1% to about 5% by weight of tableting lubricants and powder flow agents; about 0.2% to about 0.6% by weight of intensive sweeteners; or combinations thereof.

In certain embodiments, a composition comprises: sugar alcohol or a blend of sugar alcohols, a flavoring(s), an active agent(s), tableting lubricants, powder flow agents, intensive sweeteners or combinations thereof. In certain embodiments, the blend of sugar alcohols comprise one or more of: sorbitol, isomalt, xylitol, maltitol, mannitol or erythritol. In certain embodiments, the active agent(s) comprise at least one of: a chitosan, a berry extract, one or more cannabinoids, derivatives thereof or combinations thereof. In certain embodiments, the active agents based on the total weight of the composition, comprise about 1.0% to about 10.0% by weight. In certain embodiments, the one or more cannabinoids or derivatives thereof comprise a powder or oil form. In certain embodiments, the flavoring is in a liquid and/or powder form. In certain embodiments, the flavoring, based on the total weight of the composition, comprise about 2.0% to about 12.0% by weight. In certain embodiments, the sugar alcohol or a blend of sugar alcohols based on the total weight of the composition, comprise about 70.0% to about 90.0% by weight. In certain embodiments, the tableting lubricants and powder flow agents, based on the total weight of the composition, comprise about 1.5% to about 5.0% by weight.

In certain embodiments, a composition comprises: a gum base, a sugar alcohol, a blend of sugar alcohols, sweeteners, a bulk filler, a cannabinoid, chitosan, berry extracts, flavorings, tableting lubricants, powder flow agents or combinations thereof. In certain embodiments, the composition, based on the total weight of the composition, comprises: about 10% to about 80% by weight of a sugar alcohol, blend of sugar alcohols, sweetener, about 5% to about 50% by weight of bulk filler, about 0.1% to about 20% by weight of a cannabinoid or derivatives thereof, about 1% to about 10% by weight of chitosan, about 1% to about 10% by weight of berry extracts, about 0.1% to about 10% by weight of a flavor powder, about 0.1% to about 10% by weight of tableting lubricants and powder flow agents, or combinations thereof. In certain embodiments, the composition, based on the total weight of the composition, comprises: about 55% to about 70% by weight of a sugar, a sugar blend, sugar alcohol, blend of sugar alcohols, sweetener, about 5% to about 40% by weight of bulk filler, about 0.1% to about 10% by weight of a cannabinoid or derivatives thereof, about 1% to about 10% by weight of chitosan, about 1% to about 10% by weight of berry extracts, about 0.1% to about 5% by weight of a flavor powder, about 0.1% to about 5% by weight of tableting lubricants and powder flow agents, or combinations thereof. In certain embodiments, the sugar or sugar blend comprise dextrose, sucrose, fructose, glucose or combinations thereof. In certain embodiments, the sugar alcohol or sugar alcohol blend comprise: sorbitol, isomalt, xylitol, maltitol, mannitol, erythritol or combinations thereof. In certain embodiments, the sweetener comprises stevia, sucralose, monk fruit, honey or agave nectar. In certain embodiments, the tablet flow agent comprises magnesium stearate. In certain embodiments, a bulk filler comprises microcrystalline cellulose (MCC), bamboo fibers, or combinations thereof. In certain embodiments, the composition further comprises a remineralization compound, combinations of remineralization compounds or derivatives thereof. In certain embodiments, the remineralization compound or derivatives thereof comprise from about 1.0% to about 10.0% by weight, based on the total weight of the composition.

In certain embodiments, a composition consists of: a sugar, a sugar blend, sugar alcohol, a blend of sugar alcohols, sweeteners, a bulk filler, chitosan, a berry extract(s), flavorings, tableting lubricants, powder flow agents and combinations thereof. In certain embodiments, the composition, based on the total weight of the composition, consists of: about 55% to about 70% by weight of a sugar, a sugar blend, sugar alcohol, blend of sugar alcohols, sweetener, about 5% to about 40% by weight of bulk filler, about 1% to about 10% by weight of chitosan, about 1% to about 10% by weight of berry extracts, about 0.1% to about 5% by weight of a flavor powder, about 0.1% to about 5% by weight of tableting lubricants and/or powder flow agents, and combinations thereof.

In certain embodiments, a method of preventing or treating diseases or disorders associated with gingivitis and periodontitis in a subject, comprising: administering to the subject a composition comprising: chitosan, berry extract, sugar alcohol, a blend of sugar alcohols, a gum base, or combinations thereof. In certain embodiments, the composition, based on the total weight of the composition, comprises: about 0.1% to about 20% by weight cannabinoid(s), about 10% to about 80% by weight of a sugar, sugar blend, sugar alcohol, or a blend of sugar alcohols, about 5% to about 80% of a gum base. In certain embodiments, the composition further comprises: flavoring, tableting lubricants and powder flow agents, intensive sweeteners, sugar substitutes or combinations thereof. In certain embodiments, the composition, based on the total weight of the composition, further comprises: about 1% to about 20% by weight of flavoring, about 0.1% to about 10% by weight of tableting lubricants and powder flow agents, about 0.01% to about 2% by weight of intensive sweeteners. In certain embodiments, the cannabinoid(s) comprises a cannabidiol (CBD) or derivatives thereof, of about 0.1% to about 80% by weight, based on total weight of the cannabinoid(s). In certain embodiments, the cannabinoid(s) comprises about 0.1% to about 15% by weight of CBD or derivatives thereof, based on total weight of the cannabinoid(s). In certain embodiments, the sugar or sugar blend comprise dextrose, sucrose, fructose, glucose or combinations thereof. In certain embodiments, the composition further comprises a remineralization compound, combinations of remineralization compounds or derivatives thereof. In certain embodiments, n the remineralization compound or derivatives thereof comprise from about 1.0% to about 10.0% by weight, based on the total weight of the composition.

In certain embodiments, a method of preventing or treating diseases or disorders associated with gingivitis and periodontitis in a subject, comprises: administering to the subject a composition comprising: administering to a subject in need thereof, a gum-based composition for chewing, the composition comprising, based on the total weight of the composition: about 0.1% to about 20% by weight cannabinoid(s), about 10% to about 80% by weight of a sugar, sugar blend, sugar alcohol, or a blend of sugar alcohols, about 5% to about 80% of a gum base, about 1% to about 20% by weight of flavoring, about 0.1% to about 10% by weight of tableting lubricants and powder flow agents, about 0.01% to about 2% by weight of intensive sweeteners, or combinations thereof. In certain embodiments, the composition is in a gum or tablet form. In certain embodiments, the composition further comprises a remineralization compound, combinations of remineralization compounds or derivatives thereof. In certain embodiments, the remineralization compound or derivatives thereof comprise from about 1.0% to about 10.0% by weight, based on the total weight of the composition.

In certain embodiments, a composition comprises: cannabinoid(s), a sugar, sugar blend, sugar alcohol, a blend of sugar alcohols, a gum base, or combinations thereof. In certain embodiments, the composition, based on the total weight of the composition, comprises: about 0.1% to about 20% by weight cannabinoid(s), about 10% to about 80% by weight of a sugar, sugar blend, sugar alcohol, or a blend of sugar alcohols, about 5% to about 80% of a gum base. In certain embodiments, the composition further comprises: flavoring, tableting lubricants and powder flow agents, intensive sweeteners, sugar substitutes or combinations thereof. In certain embodiments, the composition, based on the total weight of the composition, further comprises: about 1% to about 20% by weight of flavoring, about 0.1% to about 10% by weight of tableting lubricants and powder flow agents, about 0.01% to about 2% by weight of intensive sweeteners. In certain embodiments, the cannabinoid(s) comprises a cannabidiol (CBD) or derivatives thereof, of about 0.1% to about 80% by weight, based on total weight of the cannabinoid(s). In certain embodiments, the sugar alcohol or sugar alcohol blend comprise: sorbitol, isomalt, xylitol, maltitol, mannitol, erythritol or combinations thereof. In certain embodiments, a sugar substitute comprises stevia, sucralose, monk fruit, honey or agave nectar.

In certain embodiments, a composition consisting of: a chitosan, a berry extract, a sugar alcohol or a blend of sugar alcohols, tableting lubricants, powder flow agents and intensive sweeteners. In certain embodiments, the composition, based on the total weight of the composition, consists of: about 70.0% to about 90.0% by weight of a sugar, sugar blend, sugar alcohol, or a blend of sugar alcohols, about 1% to about 20% of chitosan and berry extracts, about 2% to about 12% by weight of flavoring, about 1% to about 5% by weight of tableting lubricants and powder flow agents, about 0.1% to about 2% by weight of intensive sweeteners.

In certain embodiments, a composition consists of: a chitosan, a berry extract, a sugar alcohol or a blend of sugar alcohols, chitosan and berry extracts, cannabinoids or derivatives thereof, gum base, tableting lubricants, powder flow agents and intensive sweeteners. In certain embodiments, the composition, based on the total weight of the composition, consists of: about 42.0% to about 80.0% by weight of a sugar, sugar blend, sugar alcohol, or a blend of sugar alcohols, about 20% to about 30% of a gum base, about 1% to about 20% of chitosan and berry extracts, about 1% to about 10% of cannabinoids or derivatives thereof, about 2% to about 12% by weight of flavoring, about 1% to about 5% by weight of tableting lubricants and powder flow agents, about 0.2% to about 0.6% by weight of intensive sweeteners. In certain embodiments, the composition further comprises a remineralization compound, combinations of remineralization compounds or derivatives thereof. In certain embodiments, the remineralization compound or derivatives thereof comprise from about 1.0% to about 10.0% by weight, based on the total weight of the composition.

Any compositions or methods provided herein can be combined with one or more of any of the other compositions and methods provided herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used in this specification and the appended claims, the term “or” is generally employed in its sense including “and/or” unless the content clearly dictates otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, “active” is defined as the agent or agents that provide a therapeutic effect.

As used herein, the term “agent” or “active agent” is meant to encompass any molecule, chemical entity, composition, drug, therapeutic agent, chemotherapeutic agent, or biological agent capable of preventing, ameliorating, or treating a disease or other medical condition. The term includes small molecule compounds, peptides, organic or inorganic molecules, natural or synthetic compounds and the like. An agent can be assayed in accordance with the methods of the invention at any stage during clinical trials, during pre-trial testing, or following FDA-approval. In certain embodiments, the active agent is cannabinoid(s). In other embodiments, the agent is an extract of industrial hemp derived from a cultivar comprising a cannabidiol (CBD) and expressing low levels of tetrahydrocannabinol (THC). In other embodiments, the agent comprises a cannabidiol (CBD), tetrahydrocannabinol (THC) or combinations thereof.

As used herein, a “bioactive material” is defined as a material that stimulates a beneficial response from the body, particularly bonding to host bone tissue and to the formation of a calcium phosphate layer on a material surface. Bioglass (BG) is a class of bioactive material which is composed of calcium, sodium, phosphate, and silicate. They are reactive when exposed to body fluids and deposit calcium phosphate on the surface of the particles. In vitro and in vivo studies have shown that BG particles can be deposited onto dentine surfaces and subsequently occlude the dentinal tubules by inducing the formation of carbonated HAP-like materials (Earl J S, Leary R K, et al. Physical and chemical characterization of dentin surface, following treatment with NovaMin technology. J Clin Dent. 2011; 22:2-67). An example of a bioglass material, 45S5 BG, consists of 45% SiO₂, 24.5% Na₂O, 24.5% CaO, and 6% P₂O₅ in weight. It is a highly biocompatible material possessing remarkable osteoconductivity, osteoinductivity, and controllable biodegradability (Hench West J K. Biological applications of bioactive glasses. Life Chem Rep. 1996; 13:187-241). NovaMin™ is a bioactive glass containing 45% SiO₂, 24.5% Na₂O, 24.5% CaO, and 6% P₂O₅. NovaMin particles bind to the exposed dentin surface to form a protective HCA layer as well as physically fill the open tubules. When particles of the NovaMin material are exposed to an aqueous environment such as water or saliva, there is an immediate release of sodium ions, which increases the local pH leading to precipitation of the ions to form the HCA layer (Greenspan D C. NovaMin and tooth sensitivity—an overview. J Clin Dent. 2010: 21(3):61-5).

As used herein, the term “cannabinoid” refers to a chemical compound that shows direct or indirect activity at a cannabinoid receptor. There are two main cannabinoid receptors, CB1 and CB2. Other receptors that research suggests have cannabinoid activity include the GPR55 and GPR 18 receptors. The term “phytocannabinoid” refers to cannabinoids that occur in a plant species or are derived from cannabinoids occurring in a plant species. Examples of cannabinoids include, but are not limited to, Tetrahydrocannabinol (THC), Cannabidiol (CBD), Cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM). It should be understood, that compounds used in the art of pharmaceutics generally serve a variety of functions or purposes. Thus, if a compound named herein is mentioned only once or is used to define more than one term herein, its purpose or function should not be construed as being limited solely to that named purpose(s) or function(s).

As used herein, the term “chewing gum” refers to a flavored or non-flavored substance intended for chewing. The term as used herein also includes bubble gum and confectionery products containing chewing gum. In certain embodiments, chewing gum forms include, but are not limited to, tablets, sticks, solid balls, hollow balls, cut and wrap, and pellets or pillows.

As used herein a “derivative” is: a chemical substance that is related structurally to a first chemical substance and theoretically derivable from it; a compound that is formed from a similar first compound or a compound that can be imagined to arise from another first compound, if one atom of the first compound is replaced with another atom or group of atoms; a compound derived or obtained from a parent compound and containing essential elements of the parent compound; or a chemical compound that may be produced from first compound of similar structure in one or more steps.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As defined herein, an “effective” amount of a compound or agent (i.e., an effective dosage) means an amount sufficient to produce a (e.g., clinically) desirable result.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A disease or disorder is “alleviated” if the severity of a symptom of the disease or disorder, the frequency with which such a symptom is experienced by a patient, or both, is reduced.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Other aspects are described infra.

### BRIEF DESCRIPTION OF THE DRAWINGS

FIGS. 1A and 1B are graphs showing a graphical summary of the Plaque Index (FIG. 1A) and Gingival Index (FIG. 1B) comparing the Test patients versus the Control patients.

### DETAILED DESCRIPTION

Provided herein, is an innovative chewing gum that contains safe and effective active ingredients with bacteriostatic properties which prevent the growth of gingivitis bacteria. Bacteria such as, for example, Actinomyces viscosus, Actinomyces naeslundii, and Streptococcus spp., are associated with gingivitis and dental caries, whereas Porphyromonas gingivalis, Tannarella forsythia, Treponema denticola, Prevotella intemedia, and Fusobacterium nucleatum are associated with periodontopathic biofilms (Socransky S. S. et al., Periodontol 2000 “Periodontal microbial ecology” 2005; 38:135-87). The regular use of the gums embodied herein, in oral hygiene would greatly reduce the cost of dental care by preventing oral disease before it occurs and can be used by people who do not have access to conventional oral hygiene practices. The target population is any subject that suffers from gingivitis (over 100 million people) and wants a convenient, consumer-friendly, cost effective way to treat gingivitis. Other targeted specialty populations for this invention includes, children, hospitalized patients, patients who suffer from xerostomia, the elderly in community homes and deployed military personnel (who may have limited ability to clean their teeth on a regular basis).

In certain embodiments, the formulations useful in gum and other carriers, e.g. tablets, comprise chitosan and berry extracts. The synergistic combination of chitosan and berry extracts creates an environment in the oral cavity that prevents the growth of gingival bacteria (bacteriostatic). A further embodiment incorporating one or more cannabinoids provides further synergy by adding another anti-microbial and anti-inflammatory compound. A further embodiment incorporates one or more remineralization agents, for example, hydroxyapatite, to strengthen tooth enamel.

Chitosan (1-4, 2-amino-2-deoxi-b-D-glucana) is a deacetylated derivative from the biopolysaccharide chitin that is present in insect exoskeletons, crustacean shells and fungi cell walls. Chitosan has shown 1) excellent biocompatibility; 2) almost no toxicity to human beings; 3) high bioactivity and 4) antimicrobial activity. The antimicrobial activity of chitosan regarding gram-positive and gram-negative bacteria, ranges from 100 mg/l up to 100,000 mg/l and from 100 mg/l up to 1,250 mg/l for gram-negative and gram-positive bacteria, respectively. Chitosan has a significant antibacterial effect on common oral bacteria and inhibits biofilm formation which indicates a bright future in biomaterial application. There is the possibility of using chitosan as an alternative antimicrobial in toothpaste, mouthwash for oral hygiene, or health care. Chitosan has been widely used for developing drug delivery systems because of its excellent mucoadhesive properties (Sogias I. A. et al. Biomacromolecules. July; 9(7):1837-42, 2008). The mucoadhesive properties of chitosan are very beneficial for the invention since the chitosan released from the chewing gum will adhere to the walls of the oral mucosa (inside of the mouth) for long periods of time delivering bacteriostatic anti-microbial efficacy in the mouth.

Anthocyanins are a class of plant constituents collectively known as flavonoids. Anthocyanins are water-soluble and their spectral properties usually are responsible for blue, purple and red coloring of different plant parts (flowers, fruits and other plant tissues). Anthocyanins are particularly abundant in almost all types of berries. They are accumulated in fruit plants such as blackberry, red and black raspberries, blueberries, bilberries, cherries, currants, blood orange, elderberries. Berry fruits are particularly rich in different polyphenols, including anthocyanins, and their processed forms also contain anthocyanin compounds. Antimicrobial activity of berries and other anthocyanin-containing fruits is likely to be caused by multiple mechanisms and synergies because they contain various compounds including anthocyanins, weak organic acids, phenolic acids, and mixtures of their different chemical forms. Berry concentrations exhibit antimicrobial properties against important periodontal pathogens as well as S. mutans. Combining the anti-microbial properties of both chitosan and anthocyanin-rich berry extracts into a chewing gum provides a synergistic method of delivering the anti-microbial benefit directly where needed in the oral cavity.

Cannabinoids extracted from Cannabis sativa have been reported to have potential antimicrobial properties against both gram-positive and gram-negative bacterial species (Feldman, Mark et al. “Antimicrobial potential of endocannabinoid and endocannabinoid-like compounds against methicillin-resistant Staphylococcus aureus.” Scientific reports vol. 8, 1 17696. 6 Dec. 2018, doi:10.1038/s41598-018-35793-7). Cannabinoids are substantially effective in reducing the colony count of the bacterial strains of the dental plaque as compared to the well-established synthetic oral care products such as Oral B and Colgate mouth rinses (Stahl, Veronica, and Kumar Vasudevan. “Comparison of Efficacy of Cannabinoids versus Commercial Oral Care Products in Reducing Bacterial Content from Dental Plaque: A Preliminary Observation.” Cureus vol. 12, 1 e6809. 29 Jan. 2020, doi: 10.7759/cureus.6809). Further synergism for oral health is accomplished by adding one or more cannabinoids with the chitosan and berry extract blend combination.

Remineralization compounds can contribute towards restoring strength and function within tooth structure. Remineralization compounds can include, but are not limited to, hydroxyapatite (HAP), biomimetic glass and ceramic particles, like amorphous calcium sodium phosphosilicate and amorphous calcium phosphate.

Remineralizing agents have been broadly classified into the following:

i. Fluorides, e.g. sodium fluoride (NaF), sodium monofluorophosphate (Na₂FPO₃), amine fluoride (C₂₇H₆₀F₂N₂O₃), stannous fluoride (SnF₂), or combinations of these.

ii. Nonfluoride remineralizing agents
Alpha tricalcium phosphate (TCP) and beta TCP (β-TCP)
Amorphous calcium phosphate (ACP)
Casein phosphopeptides (CPPs)
CPP-ACP
Sodium calcium phosphosilicate (bioactive glass)
Xylitol
Dicalcium phosphate dehydrate (DCPD)
Nanoparticles for remineralization
   Calcium fluoride nanoparticles
   Calcium phosphate-based nanomaterials
   Nano hydroxyapatite particles (NanoHAP)
   ACP nanoparticles
   Nanobioactive glass materials

iii. Polydopamine

iv. PA

v. Oligopeptides

vi. Theobromine

vii. Arginine

viii. Self-assembling peptides

ix. Electric field-induced remineralization.

In certain embodiments, the compositions embodied herein comprise one or more remineralization agents. In certain embodiments, the compositions embodied herein comprise hydroxyapatite (HAP), fluoride, biomimetic glass and ceramic particles such as amorphous calcium sodium phosphosilicate and amorphous calcium phosphate or combinations thereof.

Fluoride: There are four mechanisms of action of fluoride. Fluoride inhibits demineralization as the fluorapatite crystals, formed by reaction with enamel apatite crystals, are more resistant to acid attack compared to HAP crystals. Second, fluoride enhances remineralization as it speeds up the growth of the new fluorapatite crystals by bringing calcium and phosphate ions together. Third, it inhibits the activity of acid producing carious bacteria, by interfering with the production of phosphoenol pyruvate (PEP) which is a key intermediate of the glycolytic pathway in bacteria. And also, the F⁻ retains on dental hard tissue, the oral mucosa and in the dental plaque to decrease demineralization and enhance remineralization (Soi S, Vinayak V, et al. J Dent Set Oral Rehabil. 2013 July-September: 19-21).

Fluoride-containing Dentifrices: Toothpastes can contain fluoride in various chemical forms mainly as sodium fluoride (NaF), sodium monofluorophosphate (Na₂FPO₃), amine fluoride (C₂₇H₆₀F₂N₂O₃), stannous fluoride (SnF₂), or combinations of these. Sodium fluoride directly provides free fluoride. Sodium monofluorophosphate is the fluoride of choice when calcium containing abrasives are used. The fluoride released is absorbed to the mineral surface, as a CaF₂ or a CaF₂-like deposit, in free or bound form. Stannous fluoride provides fluoride and stannous ions where the latter act as an antimicrobial agent. In fluoride pastes with zinc and amino acids, the basic amino acid inhibits the formation of insoluble zinc fluoride. The available zinc aids in protecting against erosion, reducing bacterial colonization and biofilm development, and provides enhanced shine to the teeth.

Calcium Phosphate Compounds: Calcium phosphate is the principal form of calcium found in bovine milk and blood. As the major components of hydroxyapatite (HA) crystals, concentrations of calcium and phosphate in saliva and plaque play a key role in influencing the tooth demineralization and remineralization processes. At equal degrees of supersaturation, an optimal rate of enamel remineralization can be obtained with a calcium/phosphate ratio of 1.6. In the plaque fluid, the Ca/P ratio is approximately 0.3 (Li X, Wang J, Joiner A, Chang J. J Dent. 2014 June; 42 Suppl 1( ): S12-20).

β-TCP: The combination of TCP with fluoride can provide greater enamel remineralization and build more acid-resistant mineral relative to fluoride alone. When it is used in toothpaste formulations, a protective barrier is created around the calcium, allowing it to coexist with the fluoride ions. During toothbrushing, TCP comes into contact with saliva, causing the barrier to dissolve and releasing calcium, phosphate, and fluoride (Hemagaran G. Remineralisation of the tooth structure—the future of dentistry. Int J PharmTech Res. 2014; 6(2):487-493).

Functionalized TCP is a low-dose calcium phosphate system that is incorporated into a single-phase aqueous or non-aqueous topical fluoride formulation. It provides a barrier that prevents premature TCP—fluoride interactions and also facilitates a targeted delivery of TCP when applied to the teeth.

Dicalcium Phosphate Dihydrate (DCPD): DCPD is a precursor for apatite that readily turns into fluorapatite in the presence of fluoride. Inclusion of DCPD in a dentifrice increases the levels of free calcium ions in the plaque fluid, and these remain elevated for up to 12 hours after brushing, when compared to conventional silica dentifrices (Kalra D D, Kalra R D, et al. J Dent Allied Sci. 2014; 3(1):24-33).

Amorphous calcium phosphate (ACP): ACP is the initial solid phase that precipitates from a highly supersaturated calcium phosphate solution and can convert readily to stable crystalline phases such as octacalcium phosphate or apatitic products. It plays as a precursor to bioapatite and as a transient phase in biomineralization.

CPP-ACP: CPP is a milk-derived protein which stabilizes clusters of ACP into CPP-ACP complexes, because at neutral pH, the “acidic motif” in CPP is a highly charged region which can bind to minerals such as Ca²⁺, Zn²⁺, Fe²⁺, Mn²⁺, and Se²⁺. CPP-ACP is a two-phase system which when mixed together reacts to form the ACP material that precipitates onto the tooth structure and elevates calcium levels in the plaque fluid. GC Tooth Mousse Plus™ and MI Paste Plus™ are formulations of CPP-ACP with incorporated fluoride to a level of 900 ppm, where the fluorides give additive effects in reducing caries experience.

Bioactive Materials: A bioactive material is defined as a material that stimulates a beneficial response from the body, particularly bonding to host bone tissue and to the formation of a calcium phosphate layer on a material surface. Bioglass (BG) is a class of bioactive material which is composed of calcium, sodium, phosphate, and silicate. They are reactive when exposed to body fluids and deposit calcium phosphate on the surface of the particles. In vitro and in vivo studies have shown that BG particles can be deposited onto dentine surfaces and subsequently occlude the dentinal tubules by inducing the formation of carbonated HAP-like materials (Jones J R. Acta Biomater. 2013 January; 9(1):4457-86. Andersson O H, Kangasniemi I. J Biomed Mater Res. 1991 August; 25(8):1019-30. Earl J S, et al., J Clin Dent. 2011; 22(3):62-7).

Nanomaterials: These materials are often added to restorative materials as inorganic fillers, such as resin composites to release calcium, phosphate, and fluoride ions for remineralization of dental hard tissues (Zhang X, Deng X, et al. Chapter Nanotechnology in Endodontics: Current and Potential Clinical Applications. Switzerland: Springer international Publishing; 2015, Remineralising Nanomaterials for Minimally Invasive Dentistry. pp, 173-193).

Calcium Fluoride Nanoparticles: The addition of nanoCaF₂ increases the cumulative fluoride release compared to the fluoride release in traditional glass ionomer cements because the CaF₂ nanoparticle (nano-CaF₂) has a 20-fold higher surface area compared with traditional glass ionomer cements (Zhang Deng X, et al 2015).

Calcium Phosphate-based Nanomaterials: Includes nanoparticles of HAP, TCP, and ACP as sources to release calcium/phosphate ions and increase the supersaturation of HAP in carious lesions (Mang X, Deng X, et al 2015).

β-TCP (Ca₃(PO₄)₂): β-TCP can be functionalized with organic and/or inorganic materials to form the so-called functionalized β-TCP (fβ-TCP).

NanoHAP Particles: Nano-sized HAP (n-HAP) is similar to the apatite crystal of tooth enamel in morphology and crystal structure and can be substituted for the natural mineral constituent of enamel for repair biomimetically. n-HAP particles with a size of 20 nm fits well with the dimensions of the nanodefects on the enamel surface caused by acidic erosion and the nanoparticles can strongly attach to the demineralized enamel surface and inhibit further acid attack.

ACP Nanoparticles: These are small spheroidal particles with a dimension in the nanoscale (40-100 nm). ACP nanoparticles, as a source of calcium and phosphate ions, have been added to composite resins, ionomer cements, and adhesives. A study using in situ caries models of humans have revealed that nanoACP-containing nanocomposites prevented demineralization at the restoration-enamel margins, producing lesser enamel mineral loss compared with the control composite. In vitro studies by Xu Zhang have confirmed that the remineralizing rate of Pchitosan—ACP complexes' treatments were significantly higher than that of fluoride treatment.

Nanobioactive Glass Materials: nanoBG particles promote mineral formation on dentin surfaces and make dentin more acid resistant (Sheng X-Y, Gong W-Y, et al. Mineral formation on dentin induced by nano-bioactive glass. CCL. 2016; 27(9):1509-1514).

Xylitol: Xylitol is a tooth friendly nonfermantable sugar alcohol which has been shown to have noncariogenic as well as cariostatic effects. It exerts the anticariogenic effects by the inactivation of S. mutans and inhibition of plaque's ability to produce acids and polysaccharides. When consumed as mints or gum, it will stimulate an increased flow of alkaline and mineral-rich saliva from small salivary glands in the palate. Increased salivary flow results in increased buffering capacity against acids and high mineral content will provide the minerals to remineralize the damaged areas of enamel.

Polydopamines: The oxidative polymerization of dopamine in aqueous solutions spontaneously forms polydopamine, mimicking DOPA, which exhibits a strong adhesive property to various substrates under wet conditions. In demineralized dentin, the collagen fibers when coated with polydopamine, remineralization was promoted, which shows that polydopamine binding to collagen fiber act as a new nucleation site that will be favorable for HA crystal growth.

Proanthocyanidin (PA): PA is a bioflavonoid, containing benzene—pyran—phenolic acid molecular nucleus. Grape seed extract (GSE) contains PA, which can form visually insoluble HA complexes when mixed with a remineralizing solution at pH 7.4.

Self-assembling Peptide: The β-sheet-forming peptides, P114, that self-assemble themselves to form three-dimensional scaffolds under defined environmental conditions have been shown to nucleate HAP. The anionic groups of the P114 side chains attract Ca⁺⁺ ions, inducing the precipitation of HAP in situ (Amaechi B T. Remineralisation therapies for initial caries lesions. Curr Oral Health Rep June. 2015; 2(2):95-101. doi: 10.1007/s40496-015-0048-9).

Polyamide: Poly(amidoamine) (PAMAM) dendrimers are known as artificial proteins which mimic the self-assembly behavior of amelogenins to form a similar structure in vitro and is used as an organic template to control the synthesis of HAP crystals. PAMAM dendrimers modified with the carboxylic acid groups (COOH) on the crystallization of HAP on etched enamel surface have proved that polyamide act as an organic template on the demineralized enamel surface to induce the formation of HAP crystals with the same structure, orientation, and mineral phase of the intact enamel in relatively short time (Chen L, Yuan H, Tang B, Liang K, Li J. Caries Res. 2015; 49(3):282-90).

Theobromine: Theobromine is a member of the xanthine family, seen in cocoa (240 mg/cup) and chocolate (1.89%), and has shown to enhance crystalline growth of the enamel (Amaechi B. T. et al., Caries Res. 2013; 47(5):399-405). In a comparative evaluation of the remineralizing potential of theobromine and sodium fluoride dentifrice by Amaechi et al., a significantly higher mineral gain was observed with theobromine and fluoride toothpaste relative to artificial saliva.

Arginine Bicarbonate: Arginine bicarbonate is an amino acid with particles of calcium carbonate, which is capable of adhering to the mineral surface. When the calcium carbonate dissolves, the released calcium is available to remineralize the mineral while the release of carbonate may give a slight local pH rise (Bennett T, van A C, et al. Monogr Oral Sci. 2013; 23:15-26. doi: 10.1159/000350458). The studies on the demineralized bovine enamel blocks by Yamashita et al. with arginine and fluoride formulations have shown that when used in combination with fluoride, arginine significantly increased fluoride uptake compared with fluoride alone, and lesions treated with arginine containing toothpaste also showed superior fluoride uptake compared with those treated with conventional fluoride toothpaste (Cheng X, Xu P, et al. Arginine promotes fluoride uptake into artificial carious lesions in vitro. Aust Dent J. 2015; 60(1):104-111. doi: 10.1111/adj.12278).

Amelogenin: The amelogenin-rich enamel organic matrix plays a critical role in regulating the growth, shape, and arrangement of HA crystals during enamel mineralization. Recombinant porcine amelogenin (rP172) was found to stabilize calcium phosphate clusters and promote the growth of hierarchically arranged enamel crystals on acid-etched lesions, significantly improving its hardness and elastic modulus (Fan Y, et al. Biomaterials 2009; 30: 478-483. Ruan Q. et al., J Mater Chem B 2015; 3: 3112-3129. Ruan Q. et al., Acta Biomater 2013; 9: 7289-7297). This biomimetic regrowth of HA crystals also generated a robust interface between the newly formed layer and native enamel ensuring efficacy and durability of restorations. An excellent low-cost and safer alternative to the full-length amelogenin is a leucine-rich amelogenin peptide that is comprised of only 56 amino acids. The non-phosphorylated leucine-rich amelogenin peptide contains only the N- and C-terminal domains of the parent amelogenin, with these domains known to be responsible for directing mineral growth and binding (Le Norcy E, et al., J Dent Res 2011; 90: 1091-1097). In vitro studies have shown treatment of enamel lesions with leucine-rich amelogenin peptide reduced lesion depth and allowed biomimetic reconstruction of enamel by promoting linear growth of mature enamel crystals along the c-axis (Bagheri G. H. et al. Biomed Mater 2015; 10: 035007. Mukherjee K. et al., J Mater Res 2016; 31: 556-563. Shafiei F. et al. Scanning 2015; 37: 179-185). The addition of mineralization inhibitors such as inorganic pyrophosphate or matrix metalloproteinase to synthetic amelogenin assemblies was able to better regulate size, shape, and orientation of a strongly adherent new mineral layer, while preventing undesirable protein occlusion within newly formed crystals [Kwak S. Y. et al. J Dent Res 2017; 96: 524-530. Prajapati S. et al. J Dent Res 2018; 97: 84-90).

### Oral Care Compositions and Methods

Gingivitis and periodontitis are common oral health problems as result of various bacteria that reside in the mouth, which if left untreated can cause other health issues inside of a person's body. The solutions to these oral care problems suffer from lack of compliance, inconvenience or high cost. Without wishing to be bound by theory, it was hypothesized that a combination of chitosan and berry extracts, each with bacteriostatic anti-microbial properties, would inhibit the growth of oral bacteria and provide a solution for gingivitis and periodontitis.

The results from experiments presented in the examples section which follows, examine the effect of chitosan and a berry extract on the plaque levels and gingival inflammation. A study in patients compared the invention with a placebo control over a three-month period. Based on these findings, the combination of chitosan and a berry extract would be an effective therapeutic for reducing gingival inflammation and dental plaque levels.

Accordingly, in embodiments the invention provides for compositions comprising therapeutically effective amounts of active agents comprising: chitosan, berry extracts, cannabinoids, remineralization compounds, anti-microbial agents, derivatives or combinations thereof.

In certain embodiments, the active agent or derivatives thereof may also be provided in microencapsulated or nanoencapsulated form or in freeze dried form. Microencapsulated, nanoencapsulated, or freeze dried cannabinoids may improve the chewing gum's taste, prevent binding with the gum matrix, control active agent release during mastication, and further improve bioavailability of the active agents.

In the chewing gum or tablet form composition according to embodiments, the active agents are provided in encapsulated form. Microencapsulation or nanoencapsulation into particles may improve bioavailability profiles of the active agents. Encapsulation of the active agents may result in particles of size 20-40 nm. Microencapsulation or nanoencapsulation may be by liposomal encapsulation, such that the active agents are present inside particles having lipid walls. Other encapsulation methods may be used.

### Chewing Gum.

In certain embodiments, the composition is a chewing gum which releases the active agent(s) during chewing. A suitable chewing gum base comprises one or more constituents including elastomers for elasticity, resins to act as binders and softeners, plasticizers to render the elastomer soft to ensure thorough blending of the gum base and flavors during shelf life. The method for manufacture of a chewing gum is exemplified in U.S. Pat. No. 9,744,128 issued Aug. 29, 2017, the contents of which are incorporated herein by reference, in its entirety. Briefly, the method comprises initially heating the gum base in ovens to melt the gum base to an internally measured temperature between 140-160° F. The ingredients, including the one or more active ingredients are combined in a mixer. The melted gum base is added to the mixer and cooled to produce a particulate mixture. The temperature of the gum base exceeds that of the mixer when first introduced, but as mixing continues it cools quickly to room temperature and forms rock-sized granular pieces. These granular pieces are then conditioned for a period of time which allows the granular pieces to dry slightly and complete the crystallization process. The pieces are conditioned for at least about 6 hours at a temperature not greater than about 75° F. and about 60% relative humidity. The pieces are then ground into a powder at room temperature with tableting excipients, and tableted. This process preserves the efficacy of the active ingredient or ingredients by avoiding exposure to high heat and extreme cold, mainly during milling that can otherwise degrade the active ingredient's efficacy.

In certain embodiments, a composition comprises therapeutically effective amounts of active agents comprising: chitosan, berry extracts, cannabinoids, remineralization compounds, anti-microbial agents, derivatives or combinations thereof.

In some embodiments, a composition based on a chewing gum comprises a therapeutically effective amount of a blend of chitosan and a berry extract, a sugar alcohol, a blend of sugar alcohols, a gum base, or combinations thereof. In certain embodiments, a therapeutically effective amount of chitosan and berry extracts comprises about 0.1% to about 20% by weight, based on the total weight of the composition. In embodiments, the cannabinoid(s) comprises a cannabidiol (CBD) or derivatives thereof of about 0.1% to about 80% by weight. In certain embodiments, the composition comprises remineralization compounds, anti-microbial agents or combinations thereof.

In other embodiments the composition comprises: about 10% to about 80% by weight based on the total weight of the composition, of a sugar, sugar blend, sugar alcohol, or a blend of sugar alcohols, and, about 5% to about 80% by weight based on the total weight of the composition, of a gum base. In certain embodiments, the composition further comprises: flavoring, tableting lubricants and powder flow agents, intensive sweeteners, sugar substitutes or combinations thereof. In certain embodiments, the composition comprises about 1% to about 20% by weight of flavoring, about 0.1% to about 10% by weight of tableting lubricants and powder flow agents, about 0.01% to about 2% by weight of intensive sweeteners and/or sugar substitutes. In embodiments, the sugar or sugar blend comprise dextrose, sucrose, fructose, glucose or combinations thereof. In other embodiments, the sugar alcohol or sugar alcohol blend comprise: sorbitol, isomalt, xylitol, maltitol, mannitol, erythritol or combinations thereof. In other embodiments, a sugar substitute comprises stevia, sucralose, monk fruit, honey or agave nectar. In certain embodiments, the chewing gum composition comprises a flavoring agent, e.g., fruity flavors, menthol flavor, eucalyptus, mint flavor, peppermint flavor, spearmint flavor, and the like. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee.

In other embodiments, the chewing gum composition comprising the cannabinoid or derivatives thereof further comprises an elastomeric base as is commonly used in chewing gum formulations that are commercially available and accepted by the consumer. The cannabinoid or the derivatives thereof may be comprised by a solid material composed of a cellulose which comprises a well-defined amount of the cannabinoid or the derivative thereof, e.g. in and/or onto voids or pores within the solid material. Accordingly, in certain embodiments, the chewing gum composition releases at least about 1% by weight to about 30% by weight, based on the total weight content of the cannabinoid or the derivative thereof in the chewing gum composition, within about one minute to about five minutes after chewing.

An elastomeric base is normally present in the chewing gum composition in an amount of about 25 to about 85% by weight, based on the total weight of the chewing gum composition.

Cannabinoids in the chewing gum composition according to embodiments may be synthetic or procured from natural source. Natural sources of cannabinoids may be from cannabis plants, hemp plants, or other organisms capable of producing cannabinoids. Organisms capable of producing cannabinoids may be genetically modified. Where cannabinoids are from natural sources, a combination of cannabinoids may be present at different concentration. The sources may be chosen such that a cannabinoid may be present as the major cannabinoid, such as CBD, CBG, or THC.

Synthetic cannabinoids may be synthesized by methods known in the art. Synthetic cannabinoids are purer, such that only one cannabinoid may be present. A combination of cannabinoids may be provided at ratios as desired. This may be done to achieve the desired concentrations for the various synthetic cannabinoids.

In certain embodiments, cannabinoids may be provided in a solid material composed of an edible solid, such as a sugar alcohol, to prevent binding with the gum base. Other solids suitable for embedding cannabinoids are contemplated, such that cannabinoids or derivatives thereof are provided within internal voids of solid materials. Alternatively, cannabinoids or derivatives thereof may be provided in a granule embedded into the gum matrix. Cannabinoids or derivatives thereof provided in these manners may improve cannabinoid release during mastication of the chewing gum according to embodiments.

Other suitable carriers which may be combined with cannabinoids before inclusion into the gum matrix may include certain celluloses such as microcrystalline cellulose derivatives, dextran, agarose, agar, pectin, alginate, xanthan, chitosan, or starch. The combination of cannabinoids and suitable carriers may result in cannabinoids being present within internal voids of these carriers.

Providing cannabinoids by combining with a suitable carrier or by providing cannabinoids in a capsule within the gum matrix may enable controlled release of cannabinoids during chewing of the chewing gum composition.

In certain embodiments, cannabinoids or derivatives thereof may also be provided in microencapsulated or nanoencapsulated form or in freeze dried form. Microencapsulated, nanoencapsulated, or freeze dried cannabinoids may improve the chewing gum's taste, prevent binding with the gum matrix, control cannabinoid release during mastication, and further improve bioavailability of the cannabinoids.

In the chewing gum composition according to embodiments, cannabinoids may be provided in encapsulated form. Microencapsulation or nanoencapsulation into particles may improve bioavailability profiles of cannabinoids. Encapsulation of cannabinoids may result in particles of size 20-40 nm. Microencapsulation or nanoencapsulation may be by liposomal encapsulation, such that the cannabinoids are present inside particles having lipid walls. Other encapsulation methods may be used.

In certain embodiments, freeze dried cannabinoids may be in solid form obtained from freezing cannabis oil containing cannabinoids and subliming other components, leaving a solid having a high cannabinoid concentration. Solid cannabinoids may be effectively incorporated into a chewing composition by combining with other suitable solid carriers and embedding the resulting solid as a granule within the chewing gum composition.

### Lozenges.

In another embodiment, the composition is a lozenge which releases the active agent(s) over a period of time, e.g. from about 3 minutes or more, once it is in the subject's mouth. The lozenges are formulated to administer a dose of about 1 to 500 mg of the active agent per application directly to the oral mucosa inside the mouth.

Accordingly, in certain embodiments, a composition for a lozenge comprises a sugar, a sugar blend, sugar alcohol, a blend of sugar alcohols, sweeteners, a bulk filler, a cannabinoid and/or derivatives thereof, flavorings, tableting lubricants, powder flow agents or combinations thereof. In certain embodiments, the composition comprises, based on the total weight of the composition: about 10% to about 80% by weight of a sugar, a sugar blend, sugar alcohol, blend of sugar alcohols, sweetener, about 5% to about 50% by weight of bulk filler, about 0.1% to about 20% by weight of a cannabinoid or derivatives thereof, about 0.1% to about 10% by weight of a flavor powder, about 0.1% to about 10% by weight of tableting lubricants and powder flow agents, or combinations thereof.

In certain embodiments, a composition for a lozenge comprises a sugar, a sugar blend, sugar alcohol, a blend of sugar alcohols, sweeteners, a bulk filler, a cannabinoid and/or derivatives thereof, flavorings, tableting lubricants, powder flow agents, chitosan, berry extracts, remineralization compounds, anti-microbial agents, derivatives or combinations thereof.

In one embodiment, the composition, based on the total weight of the composition, comprises: about 55% to about 70% by weight of a sugar, a sugar blend, sugar alcohol, blend of sugar alcohols, sweetener, about 5% to about 40% by weight of bulk filler, about 0.1% to about 10% by weight of a cannabinoid or derivatives thereof, about 0.1% to about 5% by weight of a flavor powder, about 0.1% to about 5% by weight of tableting lubricants and powder flow agents, or combinations thereof. In certain embodiments, the sugar or sugar blend comprise dextrose, sucrose, fructose, glucose or combinations thereof. In other embodiments, the sugar alcohol or sugar alcohol blend comprise: sorbitol, isomalt, xylitol, maltitol, mannitol, erythritol or combinations thereof and the sweetener comprises stevia, sucralose, monk fruit, honey or agave nectar.

In one embodiment, the tablet flow agent comprises magnesium stearate.

In other embodiments, a bulk filler comprises microcrystalline cellulose (MCC), bamboo fibers, or combinations thereof. In order to manufacture a slower versus fast-dissolving lozenge or tablet, the proportion of the bulk fillers are increased or decreased relative to the other constituents to alter the dissolution rate of the lozenge, i.e. fast-dissolving, slow dissolving etc. The bulk fillers absorb moisture quickly which creates the dissolution. Suitable fillers include celluloses and cellulose derivatives including microcrystalline cellulose, hydroxypropylcellulose and sodium carboxymethylcellulose, lactose, starches including potato starch and corn starch, carbohydrates including a cellulose derivative, e.g. hemicellulose. The cellulose derivative may be of natural origin, e.g. dextran, agarose, agar, pectin, alginate, xanthan, chitosan, starch. The cellulose derivative may also be of synthetic or semi-synthetic origin. In certain embodiments, a bulk filler comprises microcrystalline cellulose (MCC), bamboo fibers, or combinations thereof. The bulk fillers are present in the composition from about 5% to about 50% by weight of bulk filler, based on total weight of the composition. Specific examples of a suitable microcrystalline cellulose is microcrystalline cellulose comprising: AVICEL™ grades PH-100, PH-102, PH-103, PH-105, PH-112, PH-113, PH-200, PH-300, PH-302, VIVACEL™ grades 101, 102, 12, 20 and EMOCEL™ grades 50M and 90M, and the like, and mixtures thereof.

Flavors, coloring agents, spices, and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee.

### Other Formulations.

The active agents may further be formulated with acceptable excipients and/or carriers for oral consumption. The carrier may be a liquid, gel, gelcap, capsule, powder, solid tablet (coated or non-coated), tea, or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers further include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Mack Publishing Co., Easton, Pa.). Such formulations may preferably comprise from about 1 mg to 500 mg of the concentrate. Where the formulation is an oral delivery vehicle such as a capsule or tablet, the oral delivery vehicle may comprise from about 1 to 250 mg of the concentrate, 10 to 200 mg of the concentrate to 10 to 100 mg of the concentrate. A daily dosage may comprise 1, 2, 3, 4 or 5 of the oral delivery vehicles.

In other embodiments, the active agents are provided as a powder or liquid suitable for adding by the consumer to a food or beverage. For example, in some embodiments, the concentrate can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

In other embodiments, the compositions comprising the cannabinoids or derivatives thereof, further comprise one or more additional bioactive agents, phytonutrients, or nutraceutical agents to provide a dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms. Nutraceutical agents are natural, bioactive chemical compounds that have health promoting, disease preventing or medicinal properties. Examples of nutraceutical agents that may be combined with the concentrates of the present invention include, but are not limited to, resveratrol, fucoidan, Allium cepa, Allium sativum, Aloe vera, Angelica Species, Naturally Occurring Antioxidants, Aspergillus oryzae, barley grass, Bromelain, Carnitine, carotenoids and flavonoids, Catechin, Centella asiatica (Gotu kola), Coenzyme Q10, Chinese Prepared Medicines, Coleus forskohlii, Commiphora mukul, Conjugated Linoleic Acids (CLAs), Crataegus oxyacantha (Hawthorne), Curcuma longa (Turmeric), Echinacea Species (Purple Coneflower), Eleutherococcus senticosus (Siberian Ginseng), Ephedra Species, Dietary Fish Oil, Genistein, Ginkgo biloba, Glycyrrhiza (Licorice), Hypericum perforatum (St. John's Wort), Hydrastis (Goldenseal) and other Berberine-containing plants, Lactobacillus, Lobelia (Indian Tobacco), Melaleuca alternifolia, Menaquinone, Mentha piperita, n-glycolylneuraminic acid (NGNA), Panax Ginseng, Pancreatic Enzymes, Piper mythisticum, Procyanidolic Oligomers, Pygeum africanum, Quercetin, Sarsaparilla species, Serenoa repens (Saw palmetto, Sabal serrulata), Silybum marianum (Milk Thistle), Rosemary/Lemon balm, Selenite, Tabebuia avellanedae (LaPacho), Taraxacum officinale, Tanacetum parthenium (Feverfew), Taxol, Uva ursi (Bearberry), Vaccinium myrtillus (Blueberry), Valerian officinalis, Viscum album (Mistletoe), Vitamin A, Beta-Carotene and other carotenoids, and Zingiber officinale (Ginger).

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

Manufacture

The various compositions embodied herein can be manufactured using known methods.

The manufacturing of the various compositions utilizes methods of tablet compression incorporating ingredients in powder forms. The method for manufacturing the chewing gum combines the powdered ingredients with gum bases into a mixture that is then milled into a powder with a certain particle size. The powdered gum composition is compressed into a tablet using tablet presses. The method for manufacturing lozenges combines the powdered ingredients into a mixture that is then compressed into a tablet using tablet presses.

Effective Doses

Effective doses of the compositions of the present invention, for the treatment of the above described diseases, vary depending upon may different factors, including means of administration, physiological state of the patient, whether the patient is human or an animal, other medications administered, and whether treatment is prophylactic or therapeutic. Usually, the patient is a human.

The compositions can be administered on multiple occasions, wherein intervals between single dosages can be as-needed, hourly, daily, weekly, monthly, or yearly. Dosage and frequency may vary depending on the half-life of the compounds of the invention. In therapeutic applications, a relatively high dosage at relatively short intervals is sometimes required until progression of the disease is reduced or terminated, and sometimes until the patient shows partial or complete amelioration of symptoms of the disease. Thereafter, the patient can be administered a prophylactic regime.

For any active agent used in the methods of the invention, the therapeutically effective amount or dose can be estimated initially from activity assays in cell cultures and/or animals.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The following non-limiting Examples serve to illustrate selected embodiments of the invention and which do not limit the scope of the invention described in the claims. It will be appreciated that variations in proportions and alternatives in elements of the components shown will be apparent to those skilled in the art and are within the scope of embodiments of the present invention.

### Example 1: Efficacy of a Blackberry Extract and Chitosan Chewing Gum in the Reduction of Gingival Inflammation

The efficacy of a chewing gum incorporating anthocyanin-rich blackberry extract and chitosan for treating gingival inflammation and plaque levels was investigated.

Objectives: The aim of the randomized 3-month double-blinded single center study is to determine whether a chewing gum device with food additive chitosan and blackberry extract (BCE Gum), would aid in reducing gingival inflammation and plaque levels by supplementing traditional tooth brushing and flossing measures. Patients with mild to moderate gingivitis were enrolled. All enrolled subjects received baseline oral hygiene brushing instructions and a baseline clinical examination of the gingiva. The test group used the BCE Gum three times a day for a minimum 20-30 minutes duration; the control group received a placebo gum and used it in a similar manner. This study goal was to determine whether adjunctive use of BCE Gum improved gingival inflammation status. This study is referenced as ClinicalTrials.gov Identifier: NCT03237624.

Clinical Relevance: Periodontal disease remains a prevalent and preventable disease in man. Plaque bacterial biofilm remains the primary etiologic agent of disease; colonization of non-shedding tooth surfaces greatly contributes to initiation and progression of gingivitis, for example. Although there are currently available chemotherapeutic agents to supplement daily oral hygiene measures, one continuous issue is patient compliance. Chewing gum represents a unique delivery device for not only drugs and other agents, but food additives that might aid in reducing bacteria plaque colonization on tooth surfaces. For example, chitosan and chitosan-related food additive preparations, and anthocyanin-rich berry extracts, have been shown to have antimicrobial-like properties, in the disruption of bacterial colonization (bacteriostatic, not bactericidal).

Methods

| Study Arm | Intervention/Treatment |
|---|---|
| Experimental: BCE Gum | Device: BCE Gum |
| Subjects given as intervention | Chitosan preparations and |
| BCE Gum device to supplement | anthocyanin-rich berry extracts |
| oral hygiene practices. BCE Gum | have been shown to have |
| contains chitosan and blackberry | antimicrobial properties, possibly |
| extract, which are GRAS food | in the disruption of bacterial |
| ingredients. Individuals use the | colonization. These components |
| gum 20 to 30 minutes three | of a functional chewing gum will |
| times per day. Subjects will brush | supplement in the removal of |
| and floss normally twice a day. | daily build-up of dental plaque |
| | on tooth surfaces and reduce |
| | gingival inflammation. |
| | Behavioral: Oral hygiene measures |
| | Patients will be given instructions |
| | on how to brush and floss routinely |
| | (twice per day) |
| Placebo Comparator: Control | Device: Control Chewing Gum |
| chewing gum | Control chewing gum device |
| Subjects given control gum to | does not have food additive |
| supplement oral hygiene practices. | chitosan in its composition. |
| Placebo gum does not contain | Behavioral: Oral hygiene measures |
| any active ingredients. Individuals | Patients will be given instructions |
| will use this gum 20 to 30 minutes | on how to brush and floss routinely |
| three times per day. Subjects will | (twice per day) |
| brush and floss normally twice a | |
| day. | |

Results

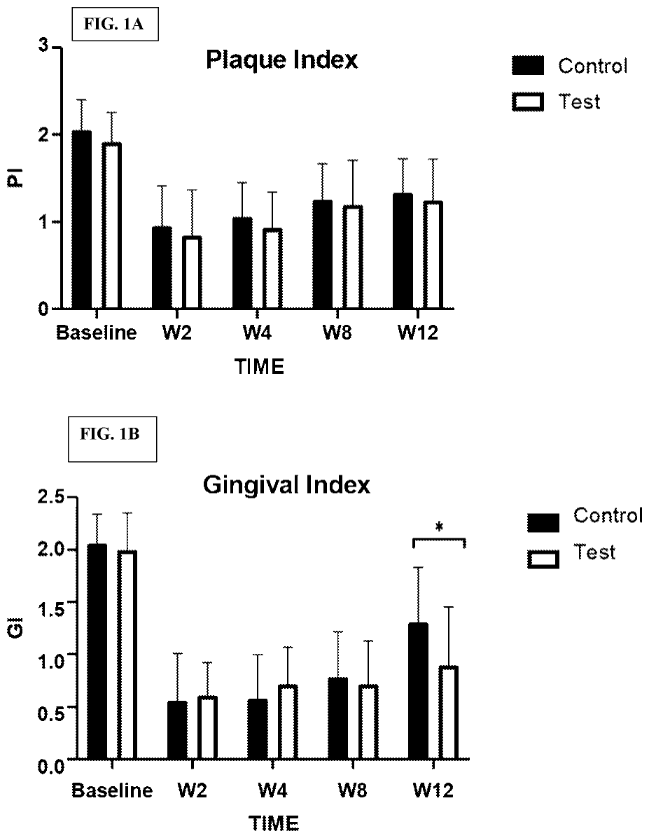

Data from Patients Studied: Thirty-four patients were studied over a 12-week period with seventeen (50%) using the blackberry extract and chitosan chewing gum (Test) and seventeen patients using placebo gum (Control) with no active ingredients. The data generated is shown in the following, (1) Table 1 shows the descriptive statistics of the Plaque Index and Gingival Index measures, and (2) FIGS. 1A and 1B are a graphical summary of the Plaque Index (FIG. 1A) and Gingival Index (FIG. 1B) comparing the Test patients versus the Control patients.

TABLE 1
Descriptive Statistics of the Plaque Index and Gingival Index Measures

| | Control | | | Test | | |
|---|---|---|---|---|---|---|
| Time | Mean | Std Dev | # of Patients | Mean | Std Dev | # of Patients |

Mean/Standard Deviation of Plaque Index Values at Timepoints

| | | | | | | |
|---|---|---|---|---|---|---|
| Base | 2.05 | 0.35 | 17 | 1.91 | 0.34 | 17 |
| 2 week | 0.95 | 0.46 | 17 | 0.84 | 0.53 | 17 |
| 4 week | 1.06 | 0.39 | 17 | 0.93 | 0.41 | 17 |
| 8 week | 1.25 | 0.41 | 17 | 1.20 | 0.51 | 17 |
| 12 week | 1.33 | 0.39 | 17 | 1.25 | 0.47 | 17 |

Mean/Standard Deviation of Gingival Index Values at Timepoints

| | | | | | | |
|---|---|---|---|---|---|---|
| Base | 2.06 | 0.28 | 17 | 2.00 | 0.35 | 17 |
| 2 week | 0.56 | 0.45 | 17 | 0.61 | 0.31 | 17 |
| 4 week | 0.58 | 0.42 | 17 | 0.72 | 0.35 | 17 |
| 8 week | 0.78 | 0.43 | 17 | 0.72 | 0.41 | 17 |
| 12 week | 1.30 | 0.53 | 17 | 0.89 | 0.56 | 17 |

### CONCLUSIONS

It was found that a chewing gum with blackberry extract and chitosan resulted in improvements in plaque levels and gingival inflammation from baseline to the end of the study. Importantly, the patients that used the Test gum had statistically significant improvements in gingival inflammation compared to those patients that used the Control (placebo) gum. These results provide evidence that the BCE gum is effective in improving the dental health of patients suffering from gingival inflammation.

The formulation shown in Table 2 shows one embodiment of a formula for a chewing gum with berry extract and chitosan.

TABLE 2

| | Weight | Optimal |
|---|---|---|
| | % Range | Weight % |
| A sugar alcohol or a | 42.4-75.3 | 62.0 |
| blend of sugar alcohols that | | |
| can include one or more of | | |
| the following: sorbitol, | | |
| isomalt, xylitol, maltitol, | | |
| mannitol or erythritol | | |
| Gum Base | 20.0-30.0 | 25.0 |
| Flavoring in liquid and | 2.0-12.0 | 3.5 |
| powder | | |
| Active ingredient— | 1.0-10.0 | 2.5 |
| chitosan | | |
| Active ingredient— berry | 1.0-10.0 | 2.5 |
| extract | | |
| Tableting lubricants and | 1.5-5.0 | 4.0 |
| powder flow agents | | |
| Intensive sweeteners | 0.2-0.6 | 0.5 |
| Total | | 100.0 |

### Example 2: Formulation for Chewing Gum with Chitosan, Berry Extract and Cannabinoids

The formulation shown in Table 3 shows one embodiment of a formula for a chewing gum with chitosan, berry extract and cannabinoids. The incorporation of cannabinoids further synergizes the anti-microbial and anti-inflammatory properties of the gum in the treatment of plaque levels and gingival inflammation in people with gingivitis and periodontitis.

TABLE 3

| | Weight % | Optimal |
|---|---|---|
| | Range | Weight % |
| A sugar alcohol or a | 42.4-75.3 | 60.5 |
| blend of sugar alcohols that | | |
| can include one or more of | | |
| the following: sorbitol, | | |
| isomalt, xylitol, maltitol, | | |
| mannitol or erythritol | | |
| Gum Base | 20.0-30.0 | 25.0 |
| Flavoring in liquid and | 2.0-12.0 | 3.5 |
| powder | | |
| Active ingredient— | 1.0-10.0 | 2.5 |
| chitosan | | |
| Active ingredient(s)— | 1.0-10.0 | 2.5 |
| berry extract | | |
| Active ingredient(s)— | 1.0-10.0 | 1.5 |
| one or more cannabinoids in | | |
| either powder or oil form | | |
| Tableting lubricants and | 1.5-5.0 | 4.0 |
| powder flow agents | | |
| Intensive sweeteners | 0.2-0.6 | 0.5 |
| Total | | 100.0 |

The formulation shown in Table 4 shows one embodiment of a formula for a chewing gum with chitosan, berry extract and hydroxyapatite. The incorporation of hydroxyapatite provides a remineralization compound to strengthen teeth in people with gingivitis and periodontitis.

TABLE 4

| | Weight % | Optimal |
|---|---|---|
| | Range | Weight % |
| A sugar alcohol or a | 42.4-75.3 | 60.5 |
| blend of sugar alcohols that | | |
| can include one or more of | | |
| the following: sorbitol, | | |
| isomalt, xylitol, maltitol, | | |
| mannitol or erythritol | | |
| Gum Base | 20.0-30.0 | 25.0 |
| Flavoring in liquid and | 2.0-12.0 | 3.5 |
| powder | | |
| Active ingredient— | 1.0-10.0 | 2.5 |
| chitosan | | |
| Active ingredient(s)— | 1.0-10.0 | 2.5 |
| berry extract | | |
| Active ingredient(s)— | 1.0-10.0 | 1.5 |
| hydroxyapatite | | |
| Tableting lubricants and | 1.5-5.0 | 4.0 |
| powder flow agents | | |
| Intensive sweeteners | 0.2-0.6 | 0.5 |
| Total | | 100.0 |

The formulation shown in Table 5 shows one embodiment of a formula for a chewing gum with chitosan and an antimicrobial agent.

TABLE 5

| | Weight % | Optimal |
|---|---|---|
| | Range | Weight % |
| A sugar alcohol or a | 42.4-75.3 | 62.0 |
| blend of sugar alcohols that | | |
| can include one or more of | | |
| the following: sorbitol, | | |
| isomalt, xylitol, maltitol, | | |
| mannitol or erythritol | | |
| Gum Base | 20.0-30.0 | 25.0 |
| Flavoring in liquid and | 2.0-12.0 | 3.5 |
| powder | | |
| Active ingredient— | 1.0-10.0 | 2.5 |
| chitosan | | |
| Active ingredient— | 1.0-10.0 | 2.5 |
| antimicrobial agent | | |
| Tableting lubricants and | 1.5-5.0 | 4.0 |
| powder flow agents | | |
| Intensive sweeteners | 0.2-0.6 | 0.5 |
| Total | | 100.0 |

### Example 3: Formulation for Lozenges

The formulation shown in Table 6 shows one embodiment of a formula for a dissolvable lozenge incorporating chitosan, berry extracts and cannabinoids. This lozenge will dissolve over a 5 to 7 minute period and deliver the active ingredients inside the dental cavity similarly to the chewing gum embodiment shown in Examples 1 and 2. This formulation may be a more practical solution for patients with gingivitis that are precluded from using chewing gum.

TABLE 6

| | Weight % | Optimal |
|---|---|---|
| | Range | Weight % |
| A sugar alcohol or a | 70.0-90.0 | 85.5 |
| blend of sugar alcohols that | | |
| can include one or more of | | |
| the following: sorbitol, | | |
| isomalt, xylitol, maltitol, | | |
| mannitol or erythritol | | |
| Flavoring in liquid and | 2.0-12.0 | 3.5 |
| powder | | |
| Active ingredient(s)— | 1.0-10.0 | 2.5 |
| chitosan | | |
| Active ingredient(s)— | 1.0-10.0 | 2.5 |
| berry extract | | |
| Active ingredient(s)— | 1.0-10.0 | 1.5 |
| one or more cannabinoids in | | |
| either powder or oil form | | |
| Tableting lubricants and | 1.5-5.0 | 4.0 |
| powder flow agents | | |
| Intensive sweeteners | 0.2-1.0 | 0.5 |
| Total | | 100.0 |

### REFERENCES

1. Carvalho M M, Thayza C M S, Emerson P S, Pedro T, Fabio S. Chitosan as an oral antimicrobial agent, Science against microbial pathogens: communicating current research and technological advances 2011. 1:542-50.
2. Zeinab Abedian, Niloofar Jenabian, Ali Akbar Moghadamnia, Ebrahim Zabihi, Hamed Tashakorian, Mandi Rajabnia, Farahnaz Sadighian, and Ali Bijani; Antibacterial activity of high-molecular-weight and low-molecular-weight chitosan upon oral pathogens, Journal of Conservative Dentistry, 2019 March-April; 22(2): 169-174.
3. loannis A. Sogias, Adrian C. Williams, and Vitaliy V. Khutoryanskiy; Why is Chitosan Mucoadhesive?; Biomacromolecules 2008, 9, 1837-1842
4. Agnieszka Cisowska, Dorota Wojnicz and Andrzej B. Hendrich; Anthocyanins as Antimicrobial Agents of Natural Plant Origin, Natural Product Communications, 2011, Vol. 6 No. 1, 149-156
5. Nohynek L, Alakomi H L, Kahkonen M P, Heinonen M, Helander I M, Oksman-Caldentey K M, Puupponen-Pimia R H. (2006) Berry phenolics: antimicrobial properties and mechanisms of action against severe human pathogens. Nutrition and Cancer, 54, 18-32.
6. Octavio A. Gonzalez, Carolina Escamilla, Robert J. Danaher, Jin Da, Jeffrey L. Ebersole, Russell J. Mumper, and Craig S. Miller, Antibacterial Effects of Blackberry Extract Target Periodontopathogens, Journal of Periodontal Research, 2013 February; 48(1), 80-86
7. Stahl V, Vasudevan K (Jan. 29, 2020) Comparison of Efficacy of Cannabinoids versus Commercial Oral Care Products in Reducing Bacterial Content from Dental Plaque: A Preliminary Observation. Cureus 12(1): e6809.
8. Feldman M, Smoum R, Mechoulam R, Steinberg D: Antimicrobial potential of endocannabinoid and endocannabinoid-like compounds against methicillin-resistant Staphylococcus aureus. Sci Rep. 2018, 8.
9. Socransky S S, Haffajee A D. Periodontal microbial ecology. Periodontol 2000. 2005; 38:135-187.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed is:

1. A composition comprising: an active ingredient, a sugar alcohol, a blend of sugar alcohols, a sweetener, flavorings, a gum base, or combinations thereof.

2. The composition of claim 1, wherein the active ingredient is an anti-microbial ingredient or combination of anti-microbial ingredients.

3. The composition of claim 1, wherein the anti-microbial ingredients comprise chitosan and berry extract(s).

4. The composition of claim 2, wherein the anti-microbial ingredients comprise from about 1.0% to about 10.0% by weight, based on the total weight of the composition.

5. The composition of claim 1, wherein the composition further comprises a cannabinoid, combinations of cannabinoids or derivatives thereof.

6. The composition of claim 5, wherein the cannabinoid(s) or derivatives thereof comprise from about 1.0% to about 10.0% by weight, based on the total weight of the composition.

7. The composition of claim 1, wherein the composition further comprises a remineralization compound, combinations of remineralization compounds or derivatives thereof.

8. The composition of claim 7, wherein the remineralization compound or derivatives thereof comprise from about 1.0% to about 10.0% by weight, based on the total weight of the composition.

9. The composition of claim 1, wherein the composition, based on the total weight of the composition, comprises:
about 40% to about 80% by weight of a sugar alcohol or a blend of sugar alcohols, or a sweetener or a combination thereof;
about 20.0% to about 30.0% by weight of a gum base;
about 2% to about 15% by weight of a flavoring in liquid or powder form;
about 1% to about 5% by weight of tableting lubricants and powder flow agents;
about 0.2% to about 0.6% by weight of intensive sweeteners; or
combinations thereof.

10. A composition comprising: sugar alcohol or a blend of sugar alcohols, a flavoring(s), an active agent(s), tableting lubricants, powder flow agents, intensive sweeteners or combinations thereof.

11. The composition of claim 10, wherein the blend of sugar alcohols comprise one or more of: sorbitol, isomalt, xylitol, maltitol, mannitol or erythritol.

12. The composition of claim 10, wherein the active agent(s) comprise at least one of: a chitosan, a berry extract, one or more cannabinoids, derivatives thereof or combinations thereof.

13. The composition of claim 10, wherein the active agents based on the total weight of the composition, comprise about 1.0% to about 10.0% by weight.

14. The composition of claim 10, wherein the one or more cannabinoids or derivatives thereof comprise a powder or oil form.

15. The composition of claim 10, wherein the flavoring is in a liquid and/or powder form.

16. The composition of claim 10, wherein the flavoring, based on the total weight of the composition, comprise about 2.0% to about 12.0% by weight.

17. The composition of claim 10, wherein the sugar alcohol or a blend of sugar alcohols based on the total weight of the composition, comprise about 70.0% to about 90.0% by weight.

18. The composition of claim 10, wherein the tableting lubricants and powder flow agents, based on the total weight of the composition, comprise about 1.5% to about 5.0% by weight.

19. A composition comprising: a gum base, a sugar alcohol, a blend of sugar alcohols, sweeteners, a bulk filler, a cannabinoid, chitosan, berry extracts, flavorings, tableting lubricants, powder flow agents or combinations thereof.

20. The composition of claim 19, wherein the composition, based on the total weight of the composition, comprises:
about 10% to about 80% by weight of a sugar alcohol, blend of sugar alcohols, sweetener,
about 5% to about 50% by weight of bulk filler,
about 0.1% to about 20% by weight of a cannabinoid or derivatives thereof,
about 1% to about 10% by weight of chitosan,
about 1% to about 10% by weight of berry extracts,
about 0.1% to about 10% by weight of a flavor powder,
about 0.1% to about 10% by weight of tableting lubricants and powder flow agents,
or combinations thereof.

21. The composition of claim 19, wherein the composition, based on the total weight of the composition, comprises:
about 55% to about 70% by weight of a sugar, a sugar blend, sugar alcohol, blend of sugar alcohols, sweetener,
about 5% to about 40% by weight of bulk filler,
about 0.1% to about 10% by weight of a cannabinoid or derivatives thereof,
about 1% to about 10% by weight of chitosan,
about 1% to about 10% by weight of berry extracts,
about 0.1% to about 5% by weight of a flavor powder,
about 0.1% to about 5% by weight of tableting lubricants and powder flow agents,
or combinations thereof.

22. The composition of claim 19, wherein the sugar or sugar blend comprise dextrose, sucrose, fructose, glucose or combinations thereof.

23. The composition of claim 19, wherein the sugar alcohol or sugar alcohol blend comprise: sorbitol, isomalt, xylitol, maltitol, mannitol, erythritol or combinations thereof.

24. The composition of claim 19, wherein the sweetener comprises stevia, sucralose, monk fruit, honey or agave nectar.

25. The composition of claim 19, wherein the tablet flow agent comprises magnesium stearate.

26. The composition of claim 19, wherein a bulk filler comprises microcrystalline cellulose (MCC), bamboo fibers, or combinations thereof.

27. The composition of claim 19, wherein the composition further comprises a remineralization compound, combinations of remineralization compounds or derivatives thereof.

28. The composition of claim 27, wherein the remineralization compound or derivatives thereof comprise from about 1.0% to about 10.0% by weight, based on the total weight of the composition.

29. A composition consisting of: a sugar, a sugar blend, sugar alcohol, a blend of sugar alcohols, sweeteners, a bulk filler, chitosan, a berry extract(s), flavorings, tableting lubricants, powder flow agents and combinations thereof.

30. The composition of claim 29, wherein the composition, based on the total weight of the composition, consists of:
about 55% to about 70% by weight of a sugar, a sugar blend, sugar alcohol, blend of sugar alcohols, sweetener,
about 5% to about 40% by weight of bulk filler,
about 1% to about 10% by weight of chitosan,
about 1% to about 10% by weight of berry extracts,
about 0.1% to about 5% by weight of a flavor powder,
about 0.1% to about 5% by weight of tableting lubricants and/or powder flow agents,
and combinations thereof.

31. A method of preventing or treating diseases or disorders associated with gingivitis and periodontitis in a subject, comprising: administering to the subject a composition comprising: chitosan, berry extract, sugar alcohol, a blend of sugar alcohols, a gum base, or combinations thereof.

32. The method of claim 31, wherein the composition, based on the total weight of the composition, comprises:
about 0.1% to about 20% by weight cannabinoid(s),
about 10% to about 80% by weight of a sugar, sugar blend, sugar alcohol, or a blend of sugar alcohols,
about 5% to about 80% of a gum base.

33. The method of claim 31, further comprising: flavoring, tableting lubricants and powder flow agents, intensive sweeteners, sugar substitutes or combinations thereof.

34. The method of claim 31, wherein the composition, based on the total weight of the composition, further comprises:
about 1% to about 20% by weight of flavoring,
about 0.1% to about 10% by weight of tableting lubricants and powder flow agents,
about 0.01% to about 2% by weight of intensive sweeteners.

35. The method of claim 31, wherein the cannabinoid(s) comprises a cannabidiol (CBD) or derivatives thereof, of about 0.1% to about 80% by weight, based on total weight of the cannabinoid(s).

36. The method of claim 35, wherein the cannabinoid(s) comprises about 0.1% to about 15% by weight of CBD or derivatives thereof, based on total weight of the cannabinoid(s).

37. The method of claim 31, wherein the sugar or sugar blend comprise dextrose, sucrose, fructose, glucose or combinations thereof.

38. The method of claim 31, wherein the composition further comprises a remineralization compound, combinations of remineralization compounds or derivatives thereof.

39. The method of claim 38, wherein the remineralization compound or derivatives thereof comprise from about 1.0% to about 10.0% by weight, based on the total weight of the composition.

40. A method of preventing or treating diseases or disorders associated with gingivitis and periodontitis in a subject, comprising: administering to the subject a composition comprising: administering to a subject in need thereof, a gum-based composition for chewing, the composition comprising, based on the total weight of the composition:
about 0.1% to about 20% by weight cannabinoid(s),
about 10% to about 80% by weight of a sugar, sugar blend, sugar alcohol, or a blend of sugar alcohols,
about 5% to about 80% of a gum base,
about 1% to about 20% by weight of flavoring,
about 0.1% to about 10% by weight of tableting lubricants and powder flow agents,
about 0.01% to about 2% by weight of intensive sweeteners,
or combinations thereof.

41. The method of claim 40, wherein the composition is in a gum or tablet form.

42. The method of claim 41, wherein the composition further comprises a remineralization compound, combinations of remineralization compounds or derivatives thereof.

43. The method of claim 42, wherein the remineralization compound or derivatives thereof comprise from about 1.0% to about 10.0% by weight, based on the total weight of the composition.

44. A composition comprising: cannabinoid(s), a sugar, sugar blend, sugar alcohol, a blend of sugar alcohols, a gum base, or combinations thereof.

45. The composition of claim 44, wherein the composition, based on the total weight of the composition, comprises:
about 0.1% to about 20% by weight cannabinoid(s),
about 10% to about 80% by weight of a sugar, sugar blend, sugar alcohol, or a blend of sugar alcohols,
about 5% to about 80% of a gum base.

46. The composition of claim 44, further comprising: flavoring, tableting lubricants and powder flow agents, intensive sweeteners, sugar substitutes or combinations thereof.

47. The composition of claim 44, wherein the composition, based on the total weight of the composition, further comprises:
about 1% to about 20% by weight of flavoring,
about 0.1% to about 10% by weight of tableting lubricants and powder flow agents,
about 0.01% to about 2% by weight of intensive sweeteners.

48. The composition of claim 44, wherein the cannabinoid(s) comprises a cannabidiol (CBD) or derivatives thereof, of about 0.1% to about 80% by weight, based on total weight of the cannabinoid(s).

49. The composition of claim 44, wherein the sugar alcohol or sugar alcohol blend comprise: sorbitol, isomalt, xylitol, maltitol, mannitol, erythritol or combinations thereof.

50. The composition of claim 44, wherein a sugar substitute comprises stevia, sucralose, monk fruit, honey or agave nectar.

51. A composition consisting of: a chitosan, a berry extract, a sugar alcohol or a blend of sugar alcohols, tableting lubricants, powder flow agents and intensive sweeteners.

52. The composition of claim 51, wherein the composition, based on the total weight of the composition, consists of:
about 70.0% to about 90.0% by weight of a sugar, sugar blend, sugar alcohol, or a blend of sugar alcohols,
about 1% to about 20% of chitosan and berry extracts,
about 2% to about 12% by weight of flavoring,
about 1% to about 5% by weight of tableting lubricants and powder flow agents,
about 0.1% to about 2% by weight of intensive sweeteners.

53. A composition consisting of: a chitosan, a berry extract, a sugar alcohol or a blend of sugar alcohols, chitosan and berry extracts, cannabinoids or derivatives thereof, gum base, tableting lubricants, powder flow agents and intensive sweeteners.

54. The composition of claim 53, wherein the composition, based on the total weight of the composition, consists of:
about 42.0% to about 80.0% by weight of a sugar, sugar blend, sugar alcohol, or a blend of sugar alcohols,
about 20% to about 30% of a gum base,
about 1% to about 20% of chitosan and berry extracts,
about 1% to about 10% of cannabinoids or derivatives thereof,
about 2% to about 12% by weight of flavoring,
about 1% to about 5% by weight of tableting lubricants and powder flow agents,
about 0.2% to about 0.6% by weight of intensive sweeteners.

55. The composition of claim 54, wherein the composition further comprises a remineralization compound, combinations of remineralization compounds or derivatives thereof.

56. The composition of claim 55, wherein the remineralization compound or derivatives thereof comprise from about 1.0% to about 10.0% by weight, based on the total weight of the composition.
